(19) United States
(12) Patent Application Publication
(10) Pub. No.: US 2025/0073501 A1
(43) Pub. Date: Mar. 6, 2025

(54) METHOD OF CONTROLLING FREQUENCY APPLIED TO PROBE OF ULTRASONIC SURGICAL DEVICE

(71) Applicant: LHBioMed Co., Ltd, Wonju-si (KR)

(72) Inventors: Han Woo LEE, Seoul (KR); Seong Soo Hong, Yongin-si (KR)

(73) Assignee: LHBioMed Co., Ltd, Wonju-si (KR)

(21) Appl. No.: 18/384,880

(22) Filed: Oct. 29, 2023

(30) Foreign Application Priority Data
Aug. 28, 2023 (KR) 10-2023-0112821

(51) Int. Cl. A61N 7/00

(52) U.S. Cl. CPC A61N 7/00; A61N 2007/0008

(57) ABSTRACT

There is provided a frequency control method including acquiring a current waveform in a form of a sinusoidal wave from a voltage waveform applied to a probe in a form of a square wave or the sinusoidal wave through a Bolt Clamped Langevin Transducer (BLT) driver, sampling the current waveform by using an analog-to-digital converter and selecting a first point and a second point, which respectively have a current phase θ−α and a current phase θ, in the current waveform, calculating the current phase θ by using a ratio between a current value a1 measured at a first point and a current value a2 measured at a second point, and adjusting a frequency of a voltage applied to the probe such that the current phase θ matches a target voltage phase.

### CROSS-REFERENCE TO RELATED APPLICATION

This application is based on and claims priority under 35 U.S.C. § 119 to Korean Patent Application No. 10-2023-0112821, filed on Aug. 28, 2023, in the Korean Intellectual Property Office, the disclosure of which is incorporated by reference herein in its entirety.

### BACKGROUND

The present disclosure relates to a frequency control method for a voltage applied to a probe of an ultrasonic surgical device, and in more detail, to a frequency control method that estimates a current phase by sampling current values at three portions of phases of a voltage applied to a probe of an ultrasonic surgical device and matches the estimated current phase to a phase of a waveform of an applied voltage.

The known liposuction often had side effects that vary from person to person, such as bumpiness after surgery, skin necrosis at a surgical site, severe swelling and bruising, extreme pain, nerve damage, and sagging skin.

Therefore, recently, ultrasonic procedures have been performed to reduce the side effects of liposuction procedures and to increase satisfaction. The ultrasonic liposuction may effectively remove fat cells by irradiating the fat layer with ultrasonic waves that vibrate tens of thousands of times per second.

FIG. 1 is a view illustrating an ultrasonic surgical device for ultrasonic liposuction.

As illustrated in FIG. 1, an ultrasonic surgical device 100 used for ultrasonic liposuction includes a handpiece 110, an ultrasonic driver installed in the handpiece 110, and a probe 120 including one end connected to the ultrasonic driver in the handpiece 110 and extending from the one end to the outside of the handpiece 110.

The probe 120 has a different thickness, length, and number of grooves depending on surgical sites and individual characteristics. In this case, the longer the length of the probe, the narrower the bandwidth ΔF of a resonator according to a center frequency.

Then, there is a problem that the prove does not vibrate or operate when a quality factor, that is, a Q factor value increases, heat is generated in the probe, and an output frequency is outside a bandwidth range during vibration.

In order to maintain constant ultrasonic output, current waveforms have to be monitored in real time, and an input drive frequency has to be adjusted from time to time through PID control such that a resonance frequency is automatically located at the center of a square wave.

The technology behind the present disclosure is disclosed in Korean Patent Publication No. 10-2021-0090078 (published on Jul. 19, 2021).

### SUMMARY

The present disclosure provides a frequency control method that estimates a current phase by sampling current values at three portions of phases of a voltage applied to a probe of an ultrasonic surgical device and matches the estimated current phase to a phase of a waveform of an applied voltage.

According to an aspect of the present disclosure, a frequency control method includes acquiring a current waveform in a form of a sinusoidal wave from a voltage waveform applied to a probe in a form of a square wave or the sinusoidal wave through a Bolt Clamped Langevin Transducer (BLT) driver, sampling the current waveform by using an analog-to-digital converter and selecting a first point and a second point, which respectively have a current phase θ−α and a current phase θ, in the current waveform, calculating the current phase θ by using a ratio between a current value a1 measured at a first point and a current value a2 measured at a second point, and adjusting a frequency of a voltage applied to the probe such that the current phase θ matches a target voltage phase.

The frequency control method may further include calculating a current value by using the current phase, estimating an applied power value by using the current value, and comparing the estimated power value with a target power value according to a power level set for the probe and adjusting an applied voltage value such that the estimated power value matches the target power value.

A ratio between the current value a1 measured at the first point and the current value a2 measured at the second point may be represented by an equation below,

$$\frac{{a}_{1}}{{a}_{2}} = \frac{A sin ( θ - α )}{A sin ( θ )} = cos ( α ) - \frac{sin ( α )}{tan ( θ )}$$

In the calculating of the current phase θ, the current phase θ is calculated by an equation below,

$$θ = {tan}^{- 1} \frac{sin ( α )}{[ cos ( α ) - \frac{{a}_{1}}{{a}_{2}} ]}$$

The frequency control method may further include selecting a third point in which a current phase is θ+α; and calculating the current phase θ by using a ratio between of a current value a3 measured at the third point and the current value a2 measured at the second point.

The ratio between the current value a3 measured at the third point and the current value a2 measured at the second point may be represented by an equation below,

$$\frac{{a}_{3}}{{a}_{2}} = \frac{A sin ( θ + α )}{A sin ( θ )} = cos ( α ) + \frac{sin ( α )}{tan ( θ )}$$

In the calculating of the current phase θ, the current phase θ may be calculated by an equation below,

$$θ = {tan}^{- 1} \frac{2 si ( α )}{[ \frac{{a}_{3}}{{a}_{2}} - \frac{{a}_{1}}{{a}_{2}} ]}$$

The first point may be located at a point where a current value increases with time, and the third point may be located at a point where a current value decreases with time.

In the adjusting of the frequency of the voltage applied to the probe, the frequency may be adjusted by using a proportional-integral-differential controller (PID) control algorithm.

In the calculating of the current phase θ, the current phase θ may be determined by an average value of calculated current phases.

### BRIEF DESCRIPTION OF THE DRAWINGS

Embodiments of the inventive concept will be more clearly understood from the following detailed description taken in conjunction with the accompanying drawings in which:

FIG. 1 is a view illustrating an ultrasonic device for ultrasonic liposuction;

FIG. 2 is a configuration diagram illustrating a frequency control device according to an embodiment of the present disclosure;

FIG. 3 is a flowchart illustrating a frequency control method using a frequency control device, according to an embodiment of the present disclosure;

FIG. 4 is a diagram illustrating step S320 illustrated in FIG. 3; and

FIG. 5 is an example diagram illustrating step S340 illustrated in FIG. 3.

### DETAILED DESCRIPTION OF THE EMBODIMENTS

Hereinafter, preferred embodiments according to the present disclosure will be described in detail with reference to the attached drawings. In this process, thicknesses of lines or sizes of components illustrated in the drawing may be exaggerated for clarity and convenience of description.

Additionally, the terms to be described below are defined in consideration of functions of the present disclosure and may change depending on the intention or custom of a user or operator. Therefore, definitions of the terms should be made based on the content throughout the present specification.

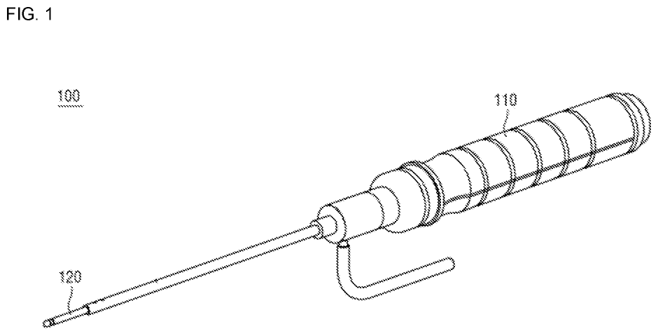

First, the Bolt Clamped Langevin Transducer (BLT) installed in an ultrasonic drive unit of an ultrasonic surgical device 100 illustrated in FIG. 1 generates stable ultrasonic waves for temperature input power (Power) and load change.

Power is transmitted most efficiently when the phase of an applied voltage matches a current, which occurs when a resonant frequency of the BLT matches the drive frequency of a driver.

Therefore, an object of an embodiment of the present disclosure is to adjust a drive frequency such that the phase of a current flowing through the BLT matches the phase of an applied voltage by using a frequency control device 200.

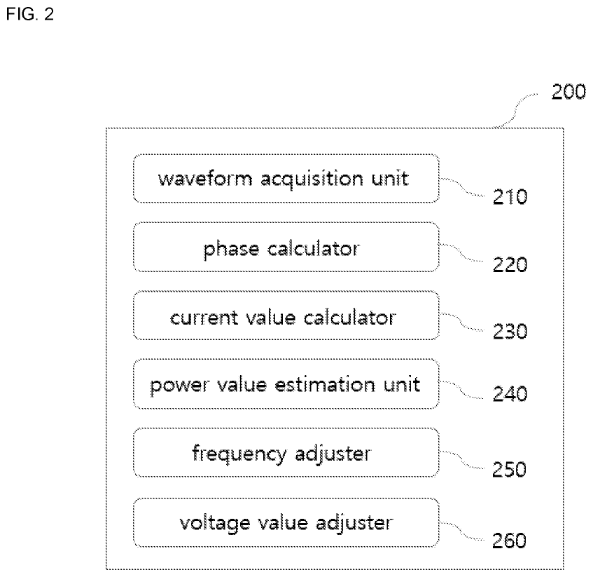

Hereinafter, the frequency control device 200 according to an embodiment of the present disclosure is described in more detail with reference to FIG. 2.

FIG. 2 is a configuration diagram illustrating a frequency control device according to an embodiment of the present disclosure.

As illustrated in FIG. 2, the frequency control device 200 according to the embodiment of the present disclosure includes a waveform acquisition unit 210, a phase calculator 220, a current value calculator 230, a power value estimation unit 240, a frequency adjuster 250, and a voltage value adjuster 260.

First, the waveform acquisition unit 210 acquires waveforms of a current applied to a BLT driver.

The phase calculator 220 selects a first point, a second point, and a third point based on the phase of a voltage currently applied to the ultrasonic surgical device 100, and calculates a current phase θ by using the current values measured at the first point, the second point, and the third point.

The current value calculator 230 calculates a current value by using the calculated current phase θ.

The power value estimation unit 240 estimates a power value by using a root mean square (RMS) value of the calculated current and a voltage and duty cycle of a drive signal.

The frequency adjuster 250 adjusts a voltage frequency applied to a probe by using a proportional-integral-differential controller (PID) control algorithm such that the calculated current phase θ matches a target voltage phase.

Finally, the voltage value adjuster 260 adjusts a voltage by comparing an estimated power value with a target power value defined according to a power level set for each probe.

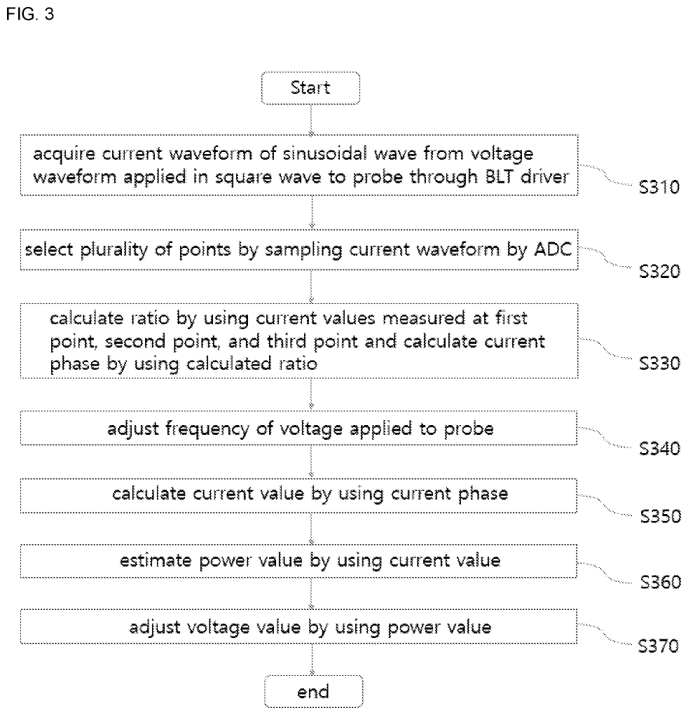

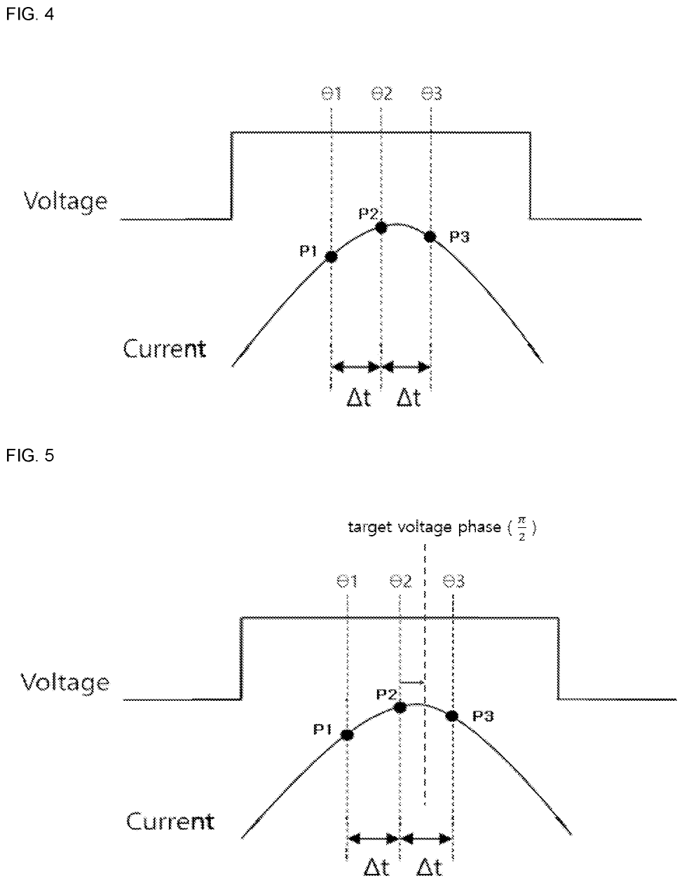

Hereinafter, a method of controlling a frequency applied to a probe of the ultrasonic surgical device 100 by using the frequency control device 200 is described in more detail with reference to FIGS. 3 and 4.

FIG. 3 is a flowchart illustrating a frequency control method using a frequency control device, according to an embodiment of the present disclosure.

As illustrated in FIG. 3, the frequency control device 200 according to an embodiment of the present disclosure acquires a current waveform in the form of a sinusoidal wave from a voltage waveform applied in the form of a square wave to the probe through a BLT driver (S310).

A current flowing through an LC circuit of the BLT driver appears in the form of a sinusoidal wave near a resonance frequency.

Accordingly, the waveform acquisition unit 210 acquires a current waveform in the form of a sinusoidal wave.

When step S310 is completed, the phase calculator 220 selects a plurality of points by sampling the acquired current waveform by using an analog-to-digital converter (ADC) (S320).

FIG. 4 is a diagram illustrating step S320 illustrated in FIG. 3.

As illustrated in FIG. 4, the phase calculator 220 samples current values by using an ADC to correspond to three portions of phases of a voltage applied to estimate the current phase. In this case, voltage and current signals and sampling positions illustrated in FIG. 4 represent an ideal case.

The sampling positions are set based on the phase of a current voltage. In this case, when a sampling interval is referred to as Δt and a drive frequency applied to the BLT driver is referred to as f_{drv}, θ1, θ2, and θ3 may be represented by Equation 1 below.

$${θ}_{1} = \frac{π}{2} - α , \\ {θ}_{2} = \frac{π}{2} , \\ {θ}_{3} = \frac{π}{2} + α , \\ where α = ( Δ t [ sec ] \times {f}_{drv} [ Hz ] * 2 π ) [ rad ] \qquad Equation 1$$

The phase calculator 220 selects a first point P1 corresponding to θ1, a second point P2 corresponding to θ2, and a third point P3 corresponding to θ3. In this case, the first point P1 is preferably located at a point where a current value increases with time, and the third point P3 is preferably located at a point where a current value decreases with time.

Next, the phase calculator 220 calculates a current phase θ by using a ratio between a current value a1 measured at the first point P1 and a current value a2 measured at the second point P2 and a ratio between a current value a3 measured at the third point P3 and the current value a2 measured at the second point P2 (S330).

Additionally, a current measured to analytically calculate the phase θ and a current value A at a voltage phase 90(π/2 [rad]) is a pure sinusoidal wave.

Then, the current value a1 at the first point P1, the current value a2 at the second point P2, and the current value a3 at the third point P3 may be represented by Equation 2 below.

$${a}_{1} = A sin ( θ - α ) , \\ {a}_{2} = A sin ( θ ) , \\ {a}_{3} = A sin ( θ + α ) , \\ where A is amplitude of current \qquad Equation 2$$

Next, the phase calculator 220 calculates a ratio between the current value a1 at the first point P1 and the current value a2 at the second point P2, which are acquired by Equation 2, by using Equation 3 below.

$$\frac{{a}_{1}}{{a}_{2}} = \frac{A sin ( θ - α )}{A sin ( θ )} = \frac{sin ( θ ) cos ( α ) - cos ( θ ) sin ( α )}{sin ( θ )} = cos ( α ) - \frac{cos ( θ ) sin ( α )}{sin ( θ )} = cos ( α ) - \frac{sin ( α )}{tan ( θ )} \qquad Equation 3$$

Here, the current value a1 at the first point P1 and the current value a2 at the second point P2 are measured values, and sin(α) and cos(α) are constants that may be calculated with a specific drive frequency f_{drv} and a sampling interval Δt, and may be represented by Equation 4 below.

$$tan ( θ ) = \frac{sin ( α )}{[ cos ( α ) - \frac{{a}_{1}}{{a}_{2}} ]} \qquad Equation 4$$

In addition, the current phase θ in the voltage phase

$$tan ( θ ) = \frac{sin ( α )}{[ cos ( α ) - \frac{{a}_{1}}{{a}_{2}} ]} \qquad Equation 4$$

may be replaced with Equation 5 below.

$$θ = {tan}^{- 1} \frac{sin ( α )}{[ cos ( α ) - \frac{{a}_{1}}{{a}_{2}} ]} \qquad Equation 5$$

In addition, the phase calculator 220 calculates the ratio between the current value a3 at the third point P3 and the current value a2 at the second point P2 acquired by Equation 2 by using Equation 6 below.

$$\frac{{a}_{3}}{{a}_{2}} = \frac{Asin ( θ + α )}{Asin ( θ )} = \frac{sin ( θ ) cos ( α ) + cos ( θ ) sin ( α )}{sin ( θ )} = cos ( α ) + \frac{cos ( θ ) sin ( α )}{sin ( θ )} = cos ( α ) + \frac{sin ( α )}{tan ( θ )} \qquad Equation 6$$

In addition, in the calculated equation 6 described above, the current value a3 at the third point P3 is a measured value, and sin(α) and cos(α) are constants that may be calculated with the specific drive frequency f_{drv} and the sampling interval Δt, and may be represented by Equation 7 below.

$$tan ( θ ) = \frac{sin ( α )}{[ \frac{{a}_{3}}{{a}_{2}} - cos ( α ) ]} \qquad Equation 7$$

In this case, two denominators of Equation 4 and Equation 7 may be represented as the same values as in Equation 8 below.

$$cos ( α ) - \frac{{a}_{1}}{{a}_{2}} = \frac{{a}_{3}}{{a}_{2}} - cos ( α ) \to \frac{1}{2} [ cos ( α ) - \frac{{a}_{1} \vert}{{a}_{2}} + \frac{{a}_{3}}{{a}_{2}} - cos ( α ) ] = \frac{1}{2} [ \frac{{a}_{3}}{{a}_{2}} - \frac{{a}_{1}}{{a}_{2}} ] \qquad Equation 8$$

Therefore, Equation 4 and Equation 7 may be replaced with Equation 9 below.

$$tan ( θ ) = \frac{2 sin ( α )}{[ \frac{{a}_{3}}{{a}_{2}} - \frac{{a}_{1}}{{a}_{2}} ]} \qquad Equation 9$$

In addition, the current phase θ in the voltage phase

$$( \frac{π}{2} )$$

may be replaced with Equation 10 below.

$$θ = {tan}^{- 1} \frac{2 si ( α )}{[ \frac{{a}_{3}}{{a}_{2}} - \frac{{a}_{1}}{{a}_{2}} ]} \qquad Equation 10$$

When step S330 is completed, the frequency adjuster 250 adjusts the frequency of a voltage applied to a probe such that the calculated current phase matches a target voltage phase (S340).

FIG. 5 is an example diagram illustrating step S340 illustrated in FIG. 3.

As illustrated in FIG. 5, the frequency adjuster 250 adjusts the frequency of a drive voltage to match the current phase θ calculated by Equation 5 or Equation 10 to a target voltage phase

$$( \frac{π}{2} ) .$$

Meanwhile, the phase θ acquired by Equation 5 may be the same as or different from the phase θ acquired by Equation 10. Hereinafter, for the sake of convenience of description, the phase θ acquired by Equation 5 is referred to as a first phase θ, and the phase θ acquired by Equation 10 is referred to as a second phase θ.

When the first phase θ is the same as the second phase θ, the frequency adjuster 250 adjusts the frequency of a voltage by using any one of the first phase θ and the second phase θ.

In addition, when the first phase θ is different from the second phase θ, the frequency adjuster 250 calculates a final phase θ by using an average value of the first phase θ and the second phase θ and adjusts the frequency of the voltage by using the calculated final phase θ.

When step S320 to step S340 are completed, the current value calculator 230 calculates a current value by using the current phase θ calculated in step S330 (S350).

Additionally, the current value calculator 230 calculates a current value A by inserting the current phase θ calculated by Equation 5 or Equation 10 into Equation 11 below.

$$A = \frac{{a}_{2}}{sin ( θ )} \qquad Equation 11$$

When step S350 is completed, the power value estimation unit 240 estimates a power value by using the calculated current value A (S360).

The power value estimation unit 240 estimates a power value P by using Equation 12 below.

$${P}_{supply} = {V}_{supply} \times {I}_{rms} \times Duty Cycle = {V}_{supply} \times \frac{A}{\sqrt{2}} \times 0.5 , where D . C . is 50 \% \qquad Equation 12$$

Here, V_{supply} represents a voltage value of the drive signal, and Iᵣₘₛ represents a current value (RMS). In this case, Iᵣₘₛ may be replaced with A/\sqrt{2}.

Finally, the voltage value adjuster 260 adjusts a voltage value by using the estimated power value P (S370).

That is, the voltage value adjuster 260 compares the estimated power value P with a target power value according to a power level set for each probe and adjusts the applied voltages to match each other.

In this way, the frequency control device according to the present disclosure may adaptively match an output frequency, which changes depending on the size or position of an ultrasonic probe, a surgical site, a distance between a probe and an object, and a surrounding environment, to a resonance frequency band.

The present disclosure is described with reference to the embodiments illustrated in the drawings, but these are merely illustrative, and those skilled in the art to which the present disclosure belongs will understand that various modifications and other equivalent embodiments may be derived therefrom. Therefore, the true technical protection scope of the present disclosure should be determined by the technical idea of the patent claims below.

What is claimed is:

1. A frequency control method comprising:
acquiring a current waveform in a form of a sinusoidal wave from a voltage waveform applied to a probe in a form of a square wave or the sinusoidal wave through a Bolt Clamped Langevin Transducer (BLT) driver;
sampling the current waveform by using an analog-to-digital converter and selecting a first point and a second point, which respectively have a current phase θ−α and a current phase θ, in the current waveform;
calculating the current phase θ by using a ratio between a current value a1 measured at a first point and a current value a2 measured at a second point; and
adjusting a frequency of a voltage applied to the probe such that the current phase θ matches a target voltage phase.

2. The frequency control method of claim 1, further comprising:
calculating a current value by using the current phase;
estimating an applied power value by using the current value; and
comparing the estimated power value with a target power value according to a power level set for the probe and adjusting an applied voltage value such that the estimated power value matches the target power value.

3. The frequency control method of claim 1, wherein a ratio between the current value a1 measured at the first point and the current value a2 measured at the second point is represented by an equation below,
$$\frac{{a}_{1}}{{a}_{2}} = \frac{Asin ( θ - α )}{A sin ( θ )} = cos ( α ) - \frac{sin ( α )}{tan ( θ )}$$

4. The frequency control method of claim 3, wherein, in the calculating of the current phase θ, the current phase θ is calculated by an equation below,
$$θ = {tan}^{- 1} \frac{sin ( α )}{[ cos ( α ) - \frac{{a}_{1}}{{a}_{2}} ]}$$

5. The frequency control method of claim 1, further comprising:
selecting a third point in which a current phase is θ+α; and
calculating the current phase θ by using a ratio between of a current value a3 measured at the third point and the current value a2 measured at the second point.

6. The frequency control method of claim 5, wherein the ratio between the current value a3 measured at the third point and the current value a2 measured at the second point is represented by an equation below,
$$\frac{{a}_{3}}{{a}_{2}} = \frac{Asin ( θ + α )}{A sin ( θ )} = cos ( α ) + \frac{sin ( α )}{tan ( θ )}$$

7. The frequency control method of claim 6, wherein, in the calculating of the current phase θ, the current phase θ is calculated by an equation below,
$$θ = {tan}^{- 1} \frac{2 si ( α )}{[ \frac{{a}_{3}}{{a}_{2}} - \frac{{a}_{1}}{{a}_{2}} ]}$$

8. The frequency control method of claim 5, wherein
the first point is located at a point where a current value increases with time, and
the third point is located at a point where a current value decreases with time.

9. The frequency control method of claim 1, wherein, in the adjusting of the frequency of the voltage applied to the probe, the frequency is adjusted by using a proportional-integral-differential controller (PID) control algorithm.

10. The frequency control method of claim 4, wherein, in the calculating of the current phase θ, the current phase θ is determined by an average value of calculated current phases.

11. The frequency control method of claim 7, wherein, in the calculating of the current phase θ, the current phase θ is determined by an average value of calculated current phases.
